(19) United States
(12) Patent Application Publication
(10) Pub. No.: US 2022/0313217 A1
(43) Pub. Date: Oct. 6, 2022

(54) METHOD AND APPARATUS FOR SHEAR WAVE GENERATION

(71) Applicant: KONINKLIJKE PHILIPS N.V., EINDHOVEN (NL)

(72) Inventor: Steven Russell Freeman, Seattle, WA (US)

(21) Appl. No.: 17/608,782

(22) PCT Filed: May 14, 2020
(86) PCT No.: PCT/EP2020/063436

§ 371 Date: Nov. 4, 2021

Related U.S. Application Data
(60) Provisional application No. 62/847,533, filed on May 14, 2019.

(51) Int. Cl. A61B 8/08; G01S 7/52; A61B 8/00

(52) U.S. Cl. CPC A61B 8/485; G01S 7/52042; G01S 7/52022; A61B 8/4488; A61B 8/54; A61B 8/461; G01S 7/52095; G01S 15/8915

(57) ABSTRACT

The present disclosure describes ultrasound systems and methods configured to interrogate the stiffness and/or elasticity of a target tissue via shear wave imaging Systems may be configured to stroboscopically transmit a plurality of push pulses into the target tissue at different focal depths. The quickly transmitted push pulses may generate shear waves that constructively interfere to form a composite shear wave. Example systems may include a beamformer configured to transmit push pulse parameters to a transducer array while receiving new push pulse parameters from a controller. Dual transmission and receipt of different push pulse parameters reconfigures the beamformer without interrupting push pulse transmission, thereby minimizing the delay between successive push pulses. Push pulses transmitted according to the disclosed methods may generate a composite shear wave configured to interrogate tissue with enhanced sensitivity across a broad depth.

### TECHNICAL FIELD

The present disclosure pertains to ultrasound systems and methods for determining tissue properties using shear waves. Particular implementations involve applying a series of ultrasound push pulses in quick succession into a target tissue to generate a composite shear wave therethrough.

### BACKGROUND

One of the long-sought goals of diagnostic imaging is precise tissue characterization. A clinician would like to use an imaging system, such as ultrasound, to identify the characteristics, e.g., benign vs. malignant, of a target tissue embodied in images thereof. One technique employed to derive tissue characteristics is ultrasound elastography, which measures the elasticity and/or stiffness of tissues in the body. For example, breast tumors or masses with high stiffness might be malignant, whereas softer and more compliant masses are likely to be benign. Ultrasound shear wave elastography, in particular, may determine the localized stiffness levels of various tissues by transmitting a “push pulse” from a transducer into a tissue, thereby generating a shear wave that propagates laterally therethrough. Tracking pulses emitted by the transducer may then be used to measure the velocity of the shear wave as it propagates, which is often proportional to the stiffness of the tissue. For example, shear wave velocity in soft tissue is typically slower than shear wave velocity in stiff tissue, assuming an identical push pulse is used to generate the shear wave in each tissue type.

Existing ultrasound elastography systems may transmit one or more push pulses at a shallow depth within a targeted tissue for a period of time, and then shift the focal zone deeper to create a shear wave that tends to propagate outward and slightly downward. Thus, to generate a comprehensive tissue scan using focal zones of multiple depths, the focus must be repeatedly adjusted, which decreases the efficacy of the overall push (and resulting shear wave) by accumulating significant transition time between each discrete pulse.

### SUMMARY

The present disclosure describes ultrasound systems and methods for determining the elasticity and/or stiffness of a target tissue via shear wave imaging. Embodiments may involve stroboscopically transmitting a plurality of push pulses into the target tissue, each push pulse having a different focal depth. Shear waves generated by the quickly transmitted push pulses may constructively interfere to form a composite shear wave having an approximately planar wavefront for interrogating a region of interest with high sensitivity. Stroboscopic transmission of push pulses having different waveform parameters may be performed with a transmit beamformer configured to receive new push pulse parameters without interrupting the ongoing transmission of a current push pulse. Receipt of new push pulse parameters during transmission of current push pulse parameters may be implemented using dual sets of shadow and active registers on the beamformer, respectively. A start signal received from a beamformer controller may initiate a transition between implementation of current push pulse parameters and a next set of push pulse parameters with very little interruption therebetween, such that the composite shear wave generated by successive pulses spans smoothly throughout the targeted tissue. In some examples, the composite shear wave may have an approximately columnar shape across a depth and lateral distance within the tissue. Various push pulse schemes may be implemented according to embodiments herein, for example at the direction of a user and/or automatically in accordance with user preferences and/or tissue dimensions. Each push pulse scheme may include various numbers of push pulses and push pulse parameters, enabling customized interrogation of a wide range of tissue types and specific regions of interest.

In accordance with principles of the present disclosure, an ultrasound imaging system for shear wave imaging may include an ultrasound transducer configured to acquire echoes in response to ultrasound pulses transmitted toward a target tissue. The system may also include a beamformer configured to transmit, from the ultrasound transducer, a current push pulse having a current focal depth in accordance with current push pulse parameters to generate a current shear wave. The beamformer may also receive next push pulse parameters for transmitting a next push pulse having a next focal depth different from the current focal depth to generate a next shear wave using a controller circuit. The current shear wave and the next shear wave may constructively interfere to generate a composite shear wave in the target tissue.

In some examples, the composite shear wave comprises an approximately columnar shape defined in part by a combined depth of the current push pulse and next push pulse. In some embodiments, the beamformer is configured to transition from the current push pulse to the next push pulse in about 250 ns to about 550 ns. In some examples, the beamformer is configured to transition from the current push pulse to the next push pulse once about every 8 μs to about 16 μs. In some embodiments, the beamformer comprises one or more active registers and one or more shadow registers configured to transmit the current push pulse parameters to the ultrasound transducer and receive the next push pulse parameters from the controller circuit, respectively. In some examples, the beamformer is configured to decrement transmit delays between the current push pulse and the next push pulse. In some embodiments, the beamformer is configured to repeat the current push pulse until the next push pulse parameters are utilized for transmitting the next push pulse.

In some examples, the controller circuit is configured to implement a push pulse scheme in accordance with a user command. The push pulse scheme may include a sequence of push pulses, each push pulse having a different focal depth. In some embodiments, the system may also include a user interface configured to display the push pulse scheme.

In some examples, the beamformer is further configured to transmit, from the ultrasound transducer, tracking pulses arranged spatially to intersect the composite shear wave at one or more locales within the target tissue. In some embodiments, the beamformer is further configured to receive, from the ultrasound transducer, echo signals that indicate the location where the tracking pulses intersected the composite shear wave. In some examples, the system also includes a tissue analysis circuit configured to determine an elasticity of the target tissue based on the echo signals.

In accordance with principles of the present disclosure, a method of shear wave imaging may involve acquiring ultrasound echoes in response to ultrasound pulses transmitted toward a target tissue; transmitting a current push pulse having a current focal depth in accordance with current push pulse parameters to generate a current shear wave; and receiving, while transmitting the current push pulse, next push pulse parameters for transmitting a next push pulse having a next focal depth different from the current focal depth to generate a next shear wave. The current shear wave and the next shear wave may constructively interfere to generate a composite shear wave in the target tissue.

In some examples, the composite shear wave comprises an approximately columnar shape defined in part by a combined depth of the current push pulse and next push pulse. Some embodiments may further involve transitioning from the current push pulse to the next push pulse once about every 8 μs to about 16 μs. Some examples may also involve decrementing transmit delays between the current push pulse and the next push pulse. Some embodiments may also involve repeating the current push pulse until the next push pulse parameters are utilized for transmitting the next push pulse. Some examples may also involve transmitting tracking pulses arranged spatially to intersect the composite shear wave at one or more locales within the target tissue.

Any of the methods described herein, or steps thereof, may be embodied in non-transitory computer-readable medium comprising executable instructions, which when executed may cause a processor circuit of a medical imaging system to perform the method or steps embodied herein.

### BRIEF DESCRIPTION OF THE DRAWINGS

FIG. 1 is a block diagram of an ultrasound imaging system constructed in accordance with principles of the present disclosure.

FIG. 2 is a diagram of a transmit beamformer constructed in accordance with principles of the present disclosure.

FIG. 3 is a diagram of a composite shear wave generated in accordance with principles of the present disclosure.

FIG. 4 is a diagram of another composite shear wave generated in accordance with principles of the present disclosure.

FIG. 5 is a flowchart showing a method performed in accordance with principles of the present disclosure.

FIG. 6 is a schematic diagram of a processor circuit in accordance with principles of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

Provided herein are ultrasound-based imaging systems configured to improve shear wave elastography by employing a transmit beamformer configured to generate a smooth shear wave through a broad depth, thereby effectively generating a shear wave in the form of a plane wave or a converging wave originating from multiple foci along the depth. The disclosed systems are configured to download new push pulse parameters, such as focusing and waveform coefficients, to the beamformer quickly without interrupting current push pulse transmission, thereby reconfiguring the beamformer while it transmits. Various push pulse parameters may be adjusted in accordance with different push pulse schemes. The push pulse parameters may include one or more properties of each discrete push pulse, or the relationship between push pulses transmitted in accordance with a given push pulse scheme. For example, the frequency of each push pulse, the waveform coefficient associated with each push pulse, the transition time between successive push pulses, the transition time between a sequence of two or more push pulses, the length of time a particular push pulse is repeated, the order and/or focal depths of successive push pulses, and/or the amplitude of each push pulse may be varied.

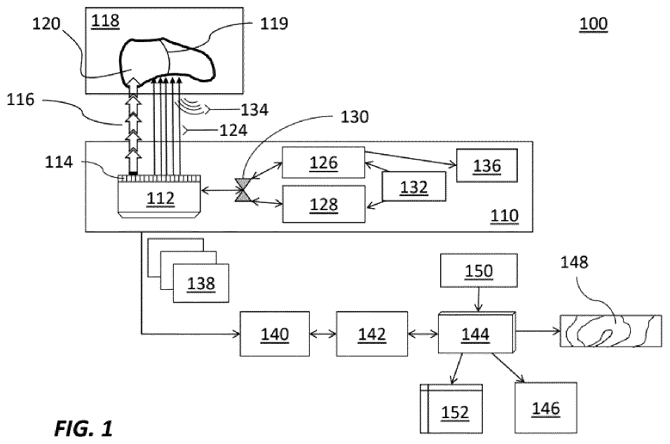

FIG. 1 shows an example ultrasound system 100 configured to perform shear wave imaging within a tissue of interest by quickly transmitting multiple push pulses having foci at different depths. The system 100 may include an ultrasound acquisition circuit 110. The ultrasound acquisition circuit 110 may include an ultrasound probe 112, a transmit beamformer 126, a multiline receive beamformer 128, a transmit/receive (T/R) switch 130, a beamformer controller circuit 132 and a signal processor circuit 136. Ultrasound acquisition circuit 110 may be constructed of hardware or a combination of hardware and software.

The ultrasound probe 112 may house an ultrasound sensor array 114. The ultrasound sensor array 114 may be configured to transmit and receive ultrasound signals. The ultrasound sensor array 114 may be configured to stroboscopically emit push pulses 116 into a target region 118. The target region 118 may be a portion of a creature, e.g., a person or animal. The creature may be alive or dead. The target region 118 may contain one or more tissue abnormalities 120, such as a tumor or stiff tissue inclusion. The target region 118 may comprise an organ, including but not limited to a human liver, pancreas, kidney, lung, heart, or brain, or an area of tissue, e.g., muscle tissue.

The push pulses 116 may be arranged to collectively form a composite shear wave 119. The composite shear waves may be created using multiple constructively interfering shear waves propagating through the target region 118. In additional or alternative embodiments, the push pulses 116 may be generated by an array other than the single ultrasound sensor array 114. For instance, in some examples, one array may be used for applying the push pulses and a different array may be used for imaging the resulting composite shear wave.

The ultrasound sensor array 114 may also be configured to transmit a plurality of tracking pulses or beams 124 into the target zone 118 to detect propagation of the shear wave 119 created by the push pulses 116. The tracking pulses 124 may be transmitted adjacent to the push pulses 116 and in some examples may be laterally-spaced with respect to the push pulses 116. In some embodiments, the tracking pulses 124 may be parallel to the push pulses 116, for example, when a linear probe is utilized to emit the tracking pulses. In other examples, the tracking pulses 124 may not be transmitted parallel to the push pulses 116. For instance, a curved probe may transmit tracking pulses 124 in a radial direction with angular separation therebetween. Such pulses may not be parallel in the Cartesian space, but they are transmitted in the same direction in the polar or cylindrical coordinate frames.

The ultrasound sensor array 114 may be coupled to the transmit beamformer 126 and the multiline receive beamformer 128 via the transmit/receive (T/R) switch 130. Coordination of transmission and reception by the beamformers 126, 128 may be controlled by a beamformer controller circuit 132. In operation, the transmit beamformer 126 may control the ultrasound sensor array 114 to transmit a series of push pulses 116 in quick succession, e.g., stroboscopically, into the target region 118, at the direction of the beamformer controller circuit 132. The transmit beamformer 126 may be configured to receive new push pulse parameters for subsequent push pulses without interrupting current push pulse transmission, thereby minimizing the transition time between successive pulses and generating the composite shear wave 119. The multiline receive beamformer 128 may produce spatially distinct receive lines (A-lines) of echo signals 134, which may be received by the ultrasound sensor array 114 and processed by filtering, noise reduction, etc. by a signal processor circuit 136. In some embodiments, the components of the acquisition circuit 110 may be configured to generate a plurality of ultrasound image frames 138 from the ultrasound echoes 134.

The system 100 may also include one or more processor circuits, such as tissue analysis circuit 140, which may be configured to determine one or more properties, e.g., stiffness and/or elasticity, of the tissue within the target region 118 based on the ultrasound image frames 138. Tissue analysis circuit 140 may be constructed of hardware, software or a combination of hardware and software.

In at least one embodiment, the system 100 also includes a display processor circuit 142 coupled with the tissue analysis circuit 140, along with a user interface 144. The display processor circuit 142 may be configured to generate ultrasound images 146 and a tissue map 148 of localized stiffness values and/or gradients. The display processor circuit 142 may be configured to generate ultrasound images 146 and/or tissue maps 148 from the image frames 138.

The user interface 144 may be configured to display the ultrasound images 146 and tissue map 148 in real time as an ultrasound scan is being performed. The user interface 144 may receive user input 150 at any time before, during or after such procedures. In some examples, the user interface 144 may be a touch screen configured to receive the user input 150 while displaying the ultrasound images 146 and/or tissue maps 148. In some examples, the ultrasound images 146 and/or tissue maps 148 displayed on the user interface 144 may be updated at every acquisition frame received and processed by the data acquisition circuit 110 during an ultrasound scan. In embodiments, the user interface 144 operating in tandem with the display processor circuit 142 may be configured to generate and display a push pulse scheme 152.

The push pulse scheme 152 may include a sequence of push pulses applied by the transmit beamformer 126, showing the focal depth of each push pulse, the order in which the pulses are transmitted, and/or the transition time between each successive pulse. The push pulse scheme 152 may include one or more push pulse parameters for each pulse, e.g., frequency and wavelength. In some examples, the push pulse scheme 152 may include an estimated shape of the composite shear wave 119 generated by the sequence of push pulses.

The configuration of the system 100 shown in FIG. 1 may vary. For example, the system 100 may be portable or stationary. In some embodiments, various portable devices, e.g., laptops, tablets, smart phones, or the like, may be used to implement one or more functions of the system 100. In examples that incorporate portable devices, the ultrasound sensor array 114 may be connectable via a USB interface. In some embodiments, one or more components shown in FIG. 1 may be combined into a single element. In some embodiments, the tissue analysis circuit 140 may be incorporated within the data acquisition circuit 110, along with the display processor circuit 142.

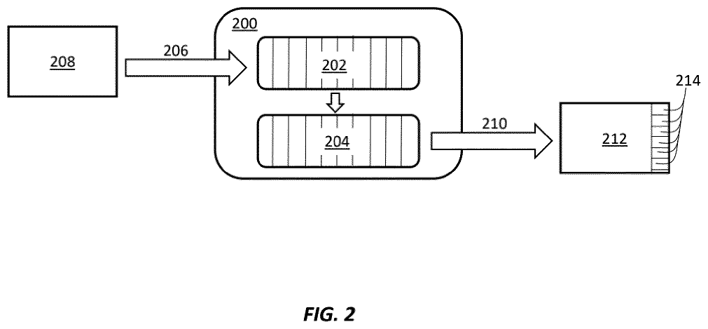

FIG. 2 is a diagram of the operations of a transmit beamformer 200 in accordance with embodiments of the present disclosure. The transmit beamformer 200 may include duplicate sets of shadow registers 202 and active registers 204. The shadow registers 202 and active registers 204 are configured to respectively receive and send different push pulse parameters simultaneously via double-buffered downloading.

In operation, the shadow registers 202 may store new or next push pulse parameters 206 from a beamformer controller circuit 208. At the same time, the active registers 204 may continue transmitting current push pulse parameters 210 to an ultrasound transducer 212. Ultrasound transducer 212 may include an ultrasound sensor array 214. Ultrasound transducer 212 may emit a push pulse into a targeted tissue in accordance with such parameters. The new push pulse parameters 206 and the current push pulse parameters 210 may specify push pulses having different focal depths. For example, the current push pulse parameters 210 may specify a push pulse having a focal depth that is more shallow within a targeted tissue than the push pulse embodied in the new push pulse parameters 206, or vice versa. The beamformer controller circuit 208 may then instruct the transmit beamformer 200 to switch to a new focal zone for the next push pulse transmission, at which point the new push pulse parameters 206 may be transferred from the shadow registers 202 to the active registers 204 and transmission of the new waveform commences via the transducer 212.

To avoid transmit delays between different elements of ultrasound sensor array 214 on a Fresnel focusing approximation may be applied, which may involve limiting all transmit delays to a single waveform period, such that transmit waveforms may complete one focus configuration and start another configuration within about one acoustic cycle. For example, transmit delays for each transmission channel may be decremented until they reach zero at which point the new push pulse parameters 206, which may be stored in shadow registers 202, may be driven to the ultrasound transducer 212 for simultaneous transmission.

In embodiments, the beamformer controller circuit 208 may be configured to repeat or loop the logic applied to generate current push pulse parameters 210, such that a particular push pulse may be repeated endlessly, or at least for a defined period of time, e.g., until the beamformer controller circuit 208 instructs the beamformer 200 to transition to a next push pulse in accordance with a next set of push pulse parameters. Thus, once a push pulse transmission begins, the transmission may continue autonomously as the beamformer 200 receives new push pulse parameters. In some examples, the beamformer controller circuit 208 may be configured to repeat or loop the logic applied to generate a series of push pulses according to a push pulse scheme, for instance, such that a composite shear wave generated from the series of push pulses may be repeatedly generated.

Configuring the transmit beamformer 200 with new push pulse parameters 206 may take between 5 μs to about 10 μs or any variation in between, depending on the specific embodiment, e.g., 7, 8, 8.7, 9 μs. The beamformer configuration time may vary. For example, if one or more push pulse parameters remain the same between successive pulses, the configuration time may be less than that required to utilize an entirely new set of push pulse parameters. Periodic reconfiguration of the transmit beamformer 200 may occur stroboscopically, such as about once every 9 μs, or in greater increments as desired, such as once every 10, 11, 12, 13, 14, 15, 16, 17, 20 μs or more, or any increment therebetween. The transition time between each successive push pulse may also vary, and may be less than a single wave-period, e.g., about 300 ns, or about 400 ns, 500 ns, 600 ns, 700 ns, 800 ns, 900 ns or any length of time therebetween. The signal embodying the new push pulse parameters 206 may be driven at a low frequency to the ultrasound transducer 212 for one or more cycles when transitioning away from a current set of push pulse parameters.

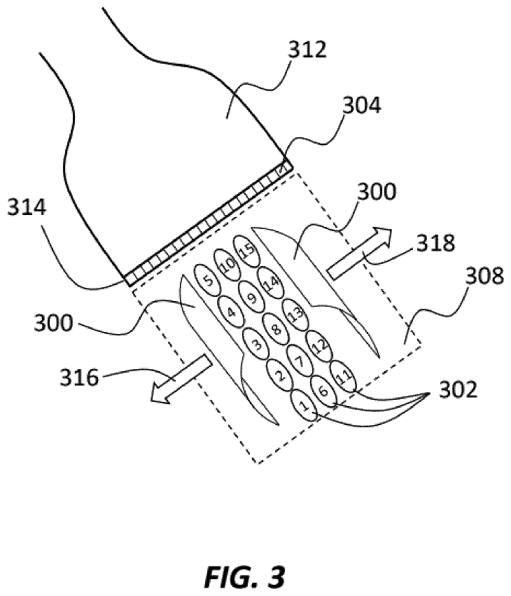

By stroboscopically reconfiguring the beamformer 200, multiple push pulses each defined by different focal depths (and/or frequencies, for example) may be smoothly transmitted with very little transition time therebetween and then released simultaneously. The rapid transmission of pulses may improve the fidelity of the resulting shear wave and may enable the generation of new shear wave shapes having enhanced sensitivity and penetration. FIG. 3, shows a series of push pulses transmitted in quick succession according to some embodiments described herein. The series of push pulses may collectively form a composite, quasi-planar or planar shear wave that is approximately cylindrical or columnar in shape due to constructive interference between the discrete shear waves generated by each push pulse. Such a columnar shear wave marks an improvement over existing mach cone shear waves, which include a plurality of spherical shear waves that do not propagate together to form a column.

FIG. 3 is a diagram of a composite shear wave 300 generated in accordance with principles of the present disclosure. The composite shear wave 300 is formed from a plurality of push pulses 302 transmitted stroboscopically from a plurality of elements 304. Elements 304 may form an ultrasound sensor array 314 on or within an ultrasound transducer 312. The composite shear wave 300 propagates radially outward through an imaging plane 308, for example in the propagation directions represented by arrows 316 and 318. Because the push pulses 302 are transmitted to different depths in quick succession and then released simultaneously, constructive interference between each adjacent wave may produce a composite shear wave 300 defined by an approximately columnar or cylindrical shape, two portions of which are shown in FIG. 3 (The foci of the push pulses 302 are not covered by the composite shear wave 300 for illustration purposes only. The composite shear wave 300 propagates radially outward from the foci of the push pulses 302). In embodiments, each of the push pulses 302 may be transmitted from a plurality of elements 304. The push pulses 302 may be transmitted successively, one after another, with very little time between the transmission of each push pulse.

Fifteen push pulses 302 are shown in this particular example; however, the number of push pulses may vary depending on the tissue being imaged, user preferences, frequency, etc., such that the number of push pulses utilized for generating a particular composite shear wave may range from 2 to 20, or more. In the embodiment shown, a push pulse 302 having a deeper focal zone (e.g., push pulse 1) is transmitted first, followed by successively shallower focal zones, e.g., up to push pulse 5. The same deep-to-shallow sequence may then be repeated one or more times in quick succession. In additional examples, push pulses having the most shallow focal zones may be transmitted first, followed by successively deeper focal zones. The number of deep-to-shallow (or shallow-to-deep) sequences may vary, along with the number of push pulses transmitted within each sequence. The lateral separation between push pulses, e.g., between push pulses 1, 6 and 11, is shown for illustration purposes only. In operation, pulses transmitted at the same focal depth may not be separated laterally. The composite shear wave 300 spans a depth of tissue defined by the plurality of push pulses, such that the entire depth may be interrogated by tracking pulses to determine the tissue elasticity and/or stiffness throughout a volume defined by the depth and lateral propagation of the composite shear wave 300.

In some embodiments, a push pulse scheme may be displayed in a manner similar to that illustrated in FIG. 3, showing the focal zones of a plurality of push pulses and the composite shear wave generated therefrom. One or more additional push pulse parameters i.e. the push pulse parameters as described above, may be displayed concurrently with the push pulse scheme. A user may adjust one or more of the parameters included in a given push pulse scheme. In some examples, a user may input a desired tissue depth and/or region of interest to be interrogated by a composite shear wave, prompting the ultrasound system, e.g., the beamformer controller circuit, to automatically generate a push pulse scheme in accordance with such instructions.

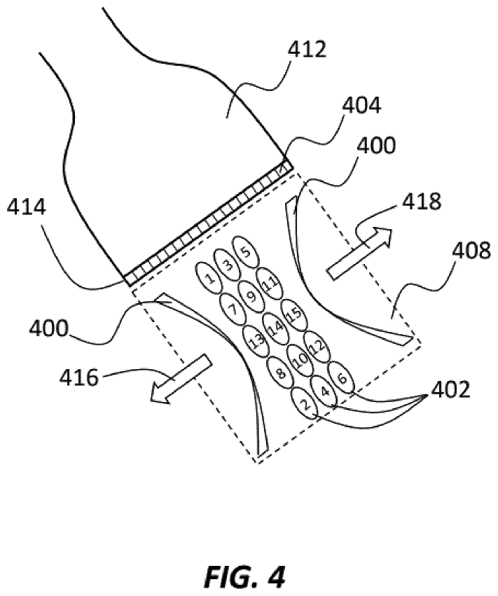

FIG. 4 is a diagram of a composite shear wave 400 generated in accordance with principles of the present disclosure. The composite shear wave 400 is formed from a plurality of push pulses 402 transmitted stroboscopically from a plurality of elements 404. Elements 404 may form an ultrasound sensor array 414 on or within an ultrasound transducer 412. The composite shear wave 400 propagates through an imaging plane 408 (shown by the dashed lines), the shear wave 400 propagating radially outward in a convex shape, for example in the propagation directions represented by arrows 416 and 418. As shown, the composite shear wave 400 has an approximately hourglass shape (two portions of which are shown) formed by constructive interference between the push pulses 402.

The composite shear wave 400 may be formed by transmitting push pulses having both deep and shallow foci (e.g., push pulses 1 and 2) in quick succession, as opposed to the push pulse scheme shown in FIG. 3, which features successive push pulses arranged spatially from deep to shallow (or shallow to deep). For instance, the most shallow and most deep push pulses 402 may be transmitted first, followed by push pulses approaching a more medial depth. In the specific example shown, push pulses 1-6 are transmitted in quick succession, alternating between deep and shallow foci. The focal depth is quickly adjusted to regions between the most deep and most shallow foci, where push pulses 7-12 are then transmitted. The focal depth may be adjusted again such that push pulses 13-15 are transmitted in quick succession. The lateral separation between push pulses, e.g., between push pulses 1, 3 and 5, is shown for illustration purposes only. In operation, pulses transmitted at the same focal depth may not be separated laterally. The tissue targeted by push pulses 1-6 may begin to relax back to its normal state after the 6^{th} push pulse is fired, at which point the shear wave generated by push pulses 1-6 may begin to propagate. The tissue targeted by push pulses 7-12 may begin to relax back to its normal state after the 12^{th} push pulse is fired, at which point the shear wave generated by push pulses 7-12 may begin to propagate. The shear wave generated by push pulses 13-15 may then begin propagating after the 15^{th} push pulse is transmitted. The composite shear wave 400 generated from each constitute shear wave may thus form the hourglass shape approximated in FIG. 4.

Near the center of the array 404, individual elements may alternate between deep and shallow-focused delays on every other element, for example, to avoid grating lobes. Outer areas of the array 404 may transmit deeply-focused pulses, for example from every other element. In various embodiments, pulses having different frequencies may be transmitted from different elements of the array 404, for example such that shallow pulses may have medium frequencies while deep pulses may have low frequencies to penetrate deeper into the targeted tissues.

By stroboscopically transmitting different sequences of push pulses having different focal depths using a quickly reconfigured beamformer according to the disclosed embodiments, composite shear waves having a variety of shapes may be generated. The time elapsed between successive pulses within a pulse sequence and/or the time elapsed between successive sequences may vary. For example, composite shear waves with convergent wave fronts may be generated by transmitting a set of alternating deep and shallow foci, quickly alternating between shallow and deep foci every 9-15 μs in the first set, followed by a successive set of alternating deep and shallow foci, e.g., about 100 ns after transmission of the first set. Successive sets of alternating foci may be transitioned closer to the center of a region of interest in embodiments, such that shear waves focused deeper may enhance the signals generated near the range of the last set of foci to fire.

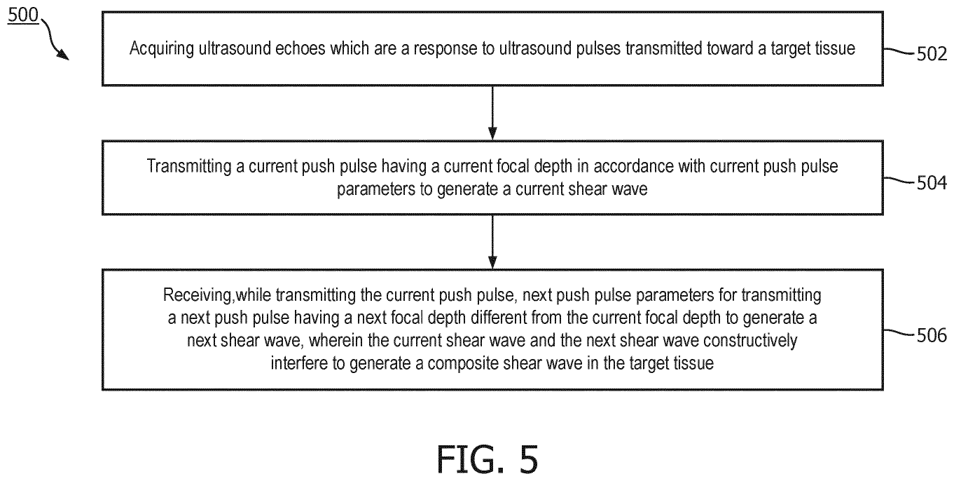

FIG. 5 is a flow diagram of a method of shear wave imaging performed in accordance with principles of the present disclosure. The example method 500 shows steps that may be utilized, in any sequence, by the systems and/or apparatuses described herein. The method 500 may be performed by an ultrasound imaging system, such as system 100, or other suitable systems including, for example, a mobile system such as LUMIFY by Koninklijke Philips N.V. (“Philips”). Additional example systems may include SPARQ and/or EPIQ, also produced by Philips.

In the embodiment shown, the method 500 begins at block 502 by “acquiring ultrasound echoes which are a response to ultrasound pulses transmitted toward a target tissue.”

At block 504, the method involves “transmitting a current push pulse having a current focal depth in accordance with current push pulse parameters to generate a current shear wave.”

At block 506, the method involves “receiving, while transmitting the current push pulse, next push pulse parameters for transmitting a next push pulse having a next focal depth different from the current focal depth to generate a next shear wave.” Pursuant to the claimed method, as further noted in block 506, “the current shear wave and the next shear wave constructively interfere to generate a composite shear wave in the target tissue.”

In various embodiments where components, systems and/or methods are implemented using a programmable device, such as a computer-based system or programmable logic, it should be appreciated that the above-described systems and methods may be implemented using any of various known or later developed programming languages, such as “C”, “C++”, “FORTRAN”, “Pascal” and the like. Accordingly, various storage media, such as magnetic computer disks, optical disks, electronic memories and the like, may be prepared that may contain information that may direct a device, such as a computer, to implement the above-described systems and/or methods. Once an appropriate device has access to the information and programs contained on the storage media, the storage media may provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein may be implemented in hardware, software and firmware. Further, the various methods and parameters are included by way of example only and not in any limiting sense. In view of this disclosure, those of ordinary skill in the art may implement the present teachings in determining their own techniques and needed equipment to affect these techniques, while remaining within the scope of the invention.

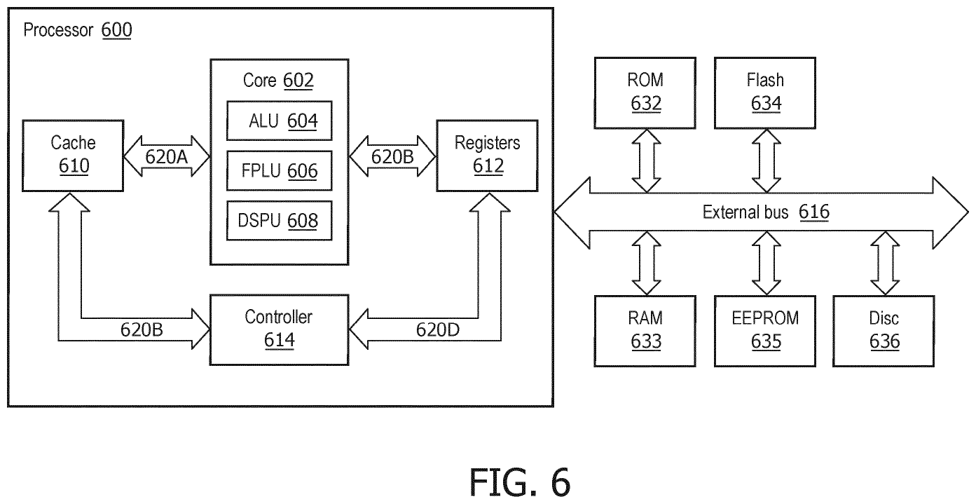

FIG. 6 is a block diagram illustrating an example processor 600 according to embodiments of the disclosure. Processor 600 may be used to implement one or more processors described herein, for example, beamformer controller circuit 132, signal processor circuit 136, tissue analysis circuit 140, and/or display processor circuit 142 shown in FIG. 1. In some examples, processor 600 may be used to implement or implement a portion of one or more components described herein, for example, motion trigger generator 450, ECG trigger generator 410, scan converter 430, multiplanar reformatter 432, and/or volume renderer 434 multiline receive beamformer 128, transmit/receive switch 130, and/or tissue analysis 140 shown in FIG. 1. Processor 600 may be any suitable processor type including, but not limited to, a microprocessor, a microcontroller, a Digital Signal Processor (DSP), a Field Programmable Gate Array (FPGA) where the FPGA has been programmed to form a processor, a Graphical Processing Unit (GPU), an Application Specific Integrated Circuit (ASIC) where the ASIC has been designed to form a processor, or a portion of custom integrated circuit or a combination thereof. The functionality of one or more of the processors described herein, including processor 600, may be incorporated into a fewer number or a single processing unit (e.g., a CPU), which may be programmed responsive to executable instruction to perform the functions described herein.

The processor 600 may include one or more cores 602 (one shown). The core 602 may include one or more arithmetic logic units (ALU) 604 (one shown). In some embodiments, the core 602 may include one or more Floating Point Logic Unit (FPLU) 606 (one shown) and/or one or more Digital Signal Processing Unit (DSPU) 608 (one shown) in addition to or instead of the one or more ALU 604.

The processor 600 may include one or more registers 612 communicatively coupled to the core 602. The registers 612 may be implemented using dedicated logic gate circuits (e.g., flip-flops) and/or any suitable memory technology. In some embodiments the registers 612 may be implemented using static memory. The register may provide data, instructions and addresses to the core 602.

In some embodiments, processor 600 may include one or more levels of cache memory 610 communicatively coupled to the core 602. The cache memory 610 may provide computer-readable instructions to the core 602 for execution. The cache memory 610 may provide data for processing by the core 602. In some embodiments, the computer-readable instructions may be provided to the cache memory 610 by a local memory, for example, local memory attached to the external bus 616. The cache memory 610 may be implemented with any suitable cache memory type, for example, Metal-Oxide Semiconductor (MOS) memory such as Static Random Access Memory (SRAM), Dynamic Random Access Memory (DRAM), and/or any other suitable memory technology.

The processor 600 may include a controller 614, which may control input to the processor 600 from other processors and/or components included in a system (e.g., transmit beamformer 200 shown in FIG. 2) and/or outputs from the processor 600 to other processors and/or components included in the system (e.g., signal processor circuit 136 shown in FIG. 1). Controller 614 may control the data paths in the ALU 604, FPLU 606 and/or DSPU 608. Controller 614 may be implemented as one or more state machines, data paths and/or dedicated control logic. The gates of controller 614 may be implemented as standalone gates, FPGA, ASIC or any other suitable technology.

The registers 612 and the cache 610 may communicate with controller 614 and core 602 via internal connections 620A, 620B, 620C and 620D. Internal connections may be implemented as a bus, multiplexor, crossbar switch, and/or any other suitable connection technology.

Inputs and outputs for the processor 600 may be provided via a bus 616, which may include one or more conductive lines. The bus 616 may be communicatively coupled to one or more components of processor 600, for example the controller 614, cache 610, and/or register 612. The bus 616 may be coupled to one or more components of the system, such as beamformer controller circuit 132, signal processor circuit 136, tissue analysis circuit 140, and/or display processor circuit 142 mentioned previously. Bus 616 may be implemented as a bus, multiplexor, crossbar switch, and/or any other suitable connection technology

The bus 616 may be coupled to one or more external memories. The external memories may include Read Only Memory (ROM) 632. ROM 632 may be a masked ROM, Electronically Programmable Read Only Memory (EPROM) 635 or any other suitable technology. The external memory may include Random Access Memory (RAM) 633. RAM 633 may be a static RAM, battery backed up static RAM, DRAM, SRAM or any other suitable technology. The external memory may include Electrically Erasable Programmable Read Only Memory (EEPROM) 635. The external memory may include Flash memory 634. The External memory may include a magnetic storage device such as disc 636. In some embodiments, the external memories may be included in a system, such as ultrasound system 100 shown in FIG. 1.

Aspects of the present technology is also described herein with reference to block diagrams and/or flowchart illustrations of methods, apparatus (systems) and/or computer program products according to the present embodiments. It is understood that blocks of the block diagrams and/or flowchart illustrations, and combinations of blocks in the block diagrams and/or flowchart illustrations, may be implemented by computer executable instructions. These computer executable instructions may be provided to a processor circuit, controller circuit or controlling unit of a general purpose computer, special purpose computer, and/or other programmable data processing apparatus to produce a machine, such that the instructions, which execute via the processor circuit of the computer and/or other programmable data processing apparatus, create means for implementing the functions/acts specified in the block diagrams and/or flowchart block or blocks.

Although the present system may have been described with particular reference to an ultrasound imaging system, it is also envisioned that the present system may be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems may be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. An ultrasound imaging system for shear wave imaging comprising:
an ultrasound transducer,
wherein the ultrasound transducer is arranged to acquire echoes,
wherein the echoes are in response to ultrasound pulses transmitted toward a target tissue; and
a beamformer,
wherein the beamformer is arranged to transmit a current push pulse,
wherein the current push pulse has a current pulse parameters,
wherein the current push pulse has a current focal depth,
wherein the current push pulse is arranged to generate a current shear wave
wherein the beamformer is arranged to receive next push pulse parameters while transmitting the current push pulse,
wherein the next push parameters are for transmitting a next push pulse,
wherein the next push pulse has a next focal depth,
wherein the next focal depth is different from the current focal depth,
wherein the next push pulse is arranged to generate a next shear wave wherein the current shear wave and the next shear wave constructively interfere to generate a composite shear wave in the target tissue.

2. The ultrasound imaging system of claim 1,
wherein the composite shear wave comprises an approximately columnar shape,
wherein the approximately columnar shape is defined in part by a combined depth of the current push pulse and next push pulse.

3. The ultrasound imaging system of claim 1, wherein the beamformer is configured to transition from the current push pulse to the next push pulse in 250 ns to 550 ns.

4. The ultrasound imaging system of 3, wherein the beamformer is configured to transition from the current push pulse to the next push pulse once every 8 μs to 16 μs.

5. The ultrasound imaging system of claim 1, wherein the beamformer comprises an active register and a shadow register configured to transmit the current push pulse parameters to the ultrasound transducer and receive the next push pulse parameters from the controller circuit, respectively.

6. The ultrasound imaging system of claim 1, wherein the beamformer is configured to decrement transmit delays between the current push pulse and the next push pulse.

7. The ultrasound imaging system of claim 1, wherein the beamformer is configured to repeat the current push pulse until the next push pulse parameters are utilized for transmitting the next push pulse.

8. The ultrasound imaging system of claim 1, wherein a controller circuit is arranged to implement a push pulse scheme in accordance with a user command.

9. The ultrasound imaging system of claim 8, wherein the push pulse scheme comprises a sequence of push pulses, each push pulse having a different focal depth.

10. The ultrasound imaging system of claim 9, further comprising a user interface configured to display the push pulse scheme.

11. The ultrasound imaging system of claim 1, wherein the beamformer is further configured to transmit, tracking pulses, wherein the tracking pulses are arranged spatially to intersect the composite shear wave at one or more locales within the target tissue.

12. The ultrasound imaging system of claim 11,
wherein the beamformer is further configured to receive, echo signals
wherein the echo signals indicate the location where the tracking pulses intersected the composite shear wave.

13. The ultrasound imaging system of claim 12, further comprising a tissue analysis circuit, wherein the tissue analysis circuit is arranged to determine an elasticity of the target tissue based on the echo signals.

14. A method of shear wave imaging, the method comprising:
acquiring ultrasound echoes, wherein the ultrasound echoes are a response to ultrasound pulses transmitted toward a target tissue;
transmitting a current push pulse,
wherein the current push pulse has a current pulse parameters,
wherein the current push pulse has a current focal depth in accordance with the current push pulse parameters,
wherein the current push pulse is arranged to generate a current shear wave; and
receiving, next push pulse parameters while transmitting the current push pulse,
wherein the next push parameters are for transmitting a next push pulse,
wherein the next push pulse has a next focal depth,
wherein the next focal depth is different from the current focal depth,
wherein the next push pulse is arranged to generate a next shear wave,
wherein the current shear wave and the next shear wave constructively interfere to generate a composite shear wave in the target tissue.

15. The method of claim 14,
wherein the composite shear wave comprises an approximately columnar shape,
wherein the approximately columnar shape is defined in part by a combined depth of the current push pulse and next push pulse.

16. The method of claim 14, further comprising transitioning from the current push pulse to the next push pulse once every 8 μs to 16 μs.

17. The method of claim 14, further comprising decrementing transmit delays between the current push pulse and the next push pulse.

18. The method of claim 14, further comprising repeating the current push pulse until the next push pulse parameters are utilized for transmitting the next push pulse.

19. The method of claim 14, further comprising transmitting tracking pulses, wherein the tracking pulses are arranged spatially to intersect the composite shear wave at one or more locales within the target tissue.

20. A non-transitory computer-readable medium comprising executable instructions, which when executed cause a processor circuit of an ultrasound imaging system to perform the method of claim 14.
